(19) United States
(12) Patent Application Publication
(10) Pub. No.: US 2025/0127855 A1
(43) Pub. Date: Apr. 24, 2025

(54) Combination Therapy for CMML and MDS

(71) Applicant: ImmuneOnco Biopharmaceuticals (Shanghai) Inc., Shanghai (CN)

(72) Inventors: Wenzhi TIAN, Shanghai (CN); Deqiang JING, Shanghai (CN); Qiying LU, Shanghai (CN); Zheng DONG, Shanghai (CN); Song LI, Shanghai (CN); Ruliang ZHANG, Shanghai (CN)

(21) Appl. No.: 18/492,925

(22) Filed: Oct. 24, 2023

Related U.S. Application Data
(63) Continuation of application No. 18/361,005, filed on Jul. 28, 2023, now abandoned.
Application No. 18/361,005 is a continuation of application No. 16/905,262, filed on Jun. 18, 2020, now abandoned.
Application No. 16/905,262 is a division of application No. 15/566,724, filed on Oct. 16, 2017, now Pat. No. 10,800,821.

(30) Foreign Application Priority Data
Apr. 24, 2015 (CN) 201510203619.7

(51) Int. Cl. A61K 38/17; A61K 31/706; A61P 35/02

(52) U.S. Cl. CPC A61K 38/1774; A61K 31/706; A61P 35/02

(57) ABSTRACT

A method of treating chronic myelomonocytic leukemia or myelodysplastic syndrome in a subject in need thereof, comprising the steps of: (a) administering intravenously to the subject for the chronic myelomonocytic leukemia about 2.0 mg/kg body weight per day of a recombinant fusion protein of SEQ ID NO: 1 in the form of a composition comprising a pharmaceutically acceptable excipient and the recombinant fusion protein, (b) about 65 minutes to about 75 minutes after completing the administering of step (a), administering subcutaneously to the subject about 75 mg/m² of azacitidine, and (c) after steps (a) and (b), repeating step (a) once weekly, and administering subcutaneously to the subject about 75 mg/m² of azacitidine once daily, wherein on the days that the subject is also having step (a) repeated, the azacitidine is administered about 65 minutes to about 75 minutes after completing the repeated administering of step (a).

### INCORPORATION BY REFERENCE

The foregoing application, and all documents cited therein or during its prosecution (“appln cited documents”) and all documents cited or referenced herein (including without limitation all literature documents, patents, published patent applications cited herein) (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference. Any Genbank sequences mentioned in this disclosure are incorporated by reference with the Genbank sequence to be that of the earliest effective filing date of this disclosure.

### SEQUENCE STATEMENT

The instant application contains a Sequence Listing XML labeled “55525-00082SequenceListingXML” which was created on Oct. 18, 2023 and is 5 bytes. The entire content of the sequence listing is incorporated herein by reference in its entirety.

### FIELD OF THE INVENTION

The disclosure relates to a pharmaceutical composition comprising a recombinant fusion protein and azacitidine, wherein the recombinant fusion protein comprises a mutated first extracellular Ig-like domain of SIRP-alpha (SIRPαD1) and a functional IgG1 heavy chain constant region. The disclosure also relates to a method for treating chronic myelomonocytic leukemia (CMML) or myelodysplastic syndrome (MDS) in a subject in need thereof using the pharmaceutical composition of the disclosure.

### BACKGROUND OF THE INVENTION

Myelodysplastic syndrome (MDS) is a group of disorders in which the bone marrow does not produce enough healthy blood cells, leading to low blood cell counts (cytopenias) and the risk of developing acute myeloid leukemia (AML). It is typically characterized by abnormalities in the blood cells, and can be diagnosed through a combination of clinical evaluation, blood tests, bone marrow biopsy, and other diagnostic tests.

Chronic myelomonocytic leukemia (CMML) is a type of blood cancer characterized by peripheral blood monocytosis and overlapping features between MDS and myeloproliferative neoplasms (MPN). In CMML, the bone marrow produces too many abnormal white blood cells, leading to a variety of symptoms and complications. CMML can be diagnosed through blood tests, bone marrow biopsy, and other diagnostic tests, and is typically classified based on the percentage of blasts in the bone marrow and the presence or absence of certain genetic mutations (Cheson BD et al., (2006) Blood. 108(2):419-25).

MDS and CMML are often studied together in clinical trials, and certain CMML patients may receive treatments that are similar to those given to MDS patients.

Hypomethylating agents, such as azacitidine, are routinely used to treat CMML patients and typically produces objective response rates (ORR) between around 39% and 54%, and complete response (CR) rates between around 10% and 18% (Costa et al., (2011) Cancer. 117(12):2690-6; Pleyer et al. (2014) Leuk Res. 38(4):475-83; Coston et al., (2019) Am J Hematol. 94(7):767-779).

Azacitidine was also approved by FDA in 2004 as the first drug to treat all subtypes of MDS, with the response rate being about 16% (Kaminskas E et al., (2005) Oncologist. 10(3):176-82). Decitabine, another hypomethylating agent, was approved by FDA in 2006 for MDS treatment. From then on, no new class of treatments have been approved in nearly 20 years for treatment of MDS, especially the high risk MDS.

Magrolimab, an anti-CD47 antibody, was once clinically tested for its efficacy in MDS and AML treatment in combination with azacitidine, and achieved quite high response rates. However, the FDA placed a partial clinical hold on the clinical trial due to suspected unexpected serious adverse reactions.

The safety problem almost bothers every conventional anti-CD47 antibody, as in addition to tumor cells, normal human red blood cells also express CD47 proteins. The binding of the anti-CD47 antibodies to red blood cells may cause serious hemolysis and anemia. To mitigate the on-target anemia, a priming dose of the anti-CD47 antibodies may be administered to eliminate aging red cells selectively while sparing younger red cells, which lack prophagocytic signals.

There is always a desperate need for more effective therapies with less adverse side effects to treatment people with MMCL or MDS.

Citation or identification of any document in this application is not an admission that such document is available as prior art to the present invention.

### SUMMARY OF THE INVENTION

The inventors of the application designed a combination therapy for CCML or MDS using a recombinant fusion protein and azacitidine. The recombinant fusion protein comprises i) a mutated SIRPαD1, as a ligand trap for CD47 neutralization, and ii) a functional IgG1 heavy chain constant region such as the Fc region, which may induce recombinant fusion protein dependent cell mediated cytotoxicity or complement dependent cytotoxicity against cells bound by the mutated SIRPαD1s. The mutated SIRPαD1 comprises a single asparagine to alanine point mutation at position 80 of the SIRPαD1, and shows higher binding capability to CD47s on Jurkat leukemia cells than the wild type SIRPαD1 and minimal binding to red blood cells (thus almost no hemagglutination).

As compared to the azacitidine monotherapy, the combination therapy of the present application produced higher complete response rate (CR) and/or higher objective response rate (ORR) in CMML and MDS patients in clinical trials. Further, the combination therapy of the present application does not require priming dose, and produced minimal hemolytic anemia and minimal grade 3 and 4 treatment-related hemolysis in the clinical trials.

Therefore, in a first aspect, the present disclosure provides a pharmaceutical composition that may comprise i) a recombinant fusion protein that may comprise a mutated SIRPαD1 and a functional IgGI heavy chain constant region, and ii) azacitidine.

The mutated SIRPαD1 may be human SIRPαD1 comprising an asparagine (Asn, N) to alanine (Ala, A) mutation at a site corresponding to the position 80 of SEQ ID NO: 2. The mutated SIRPαD1 may comprise the amino acid sequence of SEQ ID NO: 2. In certain embodiments, the mutated SIRPαD1 may consist of the amino acid sequence of SEQ ID NO: 2.

The functional IgG1 heavy chain constant region may be an IgG1 heavy chain constant region or a fragment thereof (e.g., a Fc region) that is able to bind a Fc receptor to induce recombinant fusion protein dependent cell mediated cytotoxicity or bind a complement system protein to induce complement dependent cytotoxicity. The functional IgG1 heavy chain constant region may be human IgG1 heavy chain constant region, or the Fc region thereof. The functional IgG1 heavy chain constant region may be the Fc region of human IgG1 heavy chain constant region, comprising e.g., the amino acid sequence of SEQ ID NO: 3. The functional IgG1 heavy chain constant region may, in certain embodiments, consist of the amino acid sequence of SEQ ID NO: 3.

The recombinant fusion protein may comprise, from the N terminus to the C terminus, the mutated SIRPαD1 and the functional IgGI heavy chain constant region. The recombinant fusion protein may comprise the amino acid sequence of SEQ ID NO: 1, and may consist of the amino acid sequence of SEQ ID NO: 1 in certain embodiments.

In a second aspect, the disclosure may provide a method of treating chronic myelomonocytic leukemia in a subject in need thereof, which may comprise the steps of:
(a) administering intravenously to the subject for the chronic myelomonocytic leukemia about 2.0 mg/kg body weight per day of a recombinant fusion protein in the form of a composition comprising a pharmaceutically acceptable excipient and the recombinant fusion protein,
(b) administering subcutaneously to the subject about 75 mg/m² of azacitidine, and
(c) after steps (a) and (b), repeating step (a) once weekly, and administering subcutaneously to the subject about 75 mg/m² of azacitidine once daily.

The step (b) may be performed about 65 minutes to about 75 minutes after completing the administering of step (a).

In step (c), the azacitidine may be administered about 65 minutes to about 75 minutes after completing the repeated administering of step (a) on the days that the subject is also having step (a) repeated.

The recombinant fusion protein may comprise a mutated SIRPαD1 and a functional IgG1 heavy chain constant region. The mutated SIRPαD1 may be human SIRPαD1 comprising an asparagine (Asn, N) to alanine (Ala, A) mutation at a site corresponding to the position 80 of SEQ ID NO: 2. The mutated SIRPαD1 may comprise the amino acid sequence of SEQ ID NO: 2. In certain embodiments, the mutated SIRPαD1 may consist of the amino acid sequence of SEQ ID NO: 2. The functional IgG1 heavy chain constant region may be an IgG1 heavy chain constant region or a fragment thereof (e.g., a Fc region) that is able to bind a Fc receptor to induce recombinant fusion protein dependent cell mediated cytotoxicity or bind a complement system protein to induce complement dependent cytotoxicity. The functional IgG1 heavy chain constant region may be human IgG1 heavy chain constant region, or the Fc region thereof. The functional IgG1 heavy chain constant region may be the Fc region of human IgG1 heavy chain constant region, comprising e.g., the amino acid sequence of SEQ ID NO: 3. The functional IgG1 heavy chain constant region may, in certain embodiments, consist of the amino acid sequence of SEQ ID NO: 3. The recombinant fusion protein may comprise, from the N terminus to the C terminus, the mutated SIRPαD1 and the functional IgG1 heavy chain constant region. The recombinant fusion protein may comprise the amino acid sequence of SEQ ID NO: 1,and may consist of the amino acid sequence of SEQ ID NO: 1 in certain embodiments.

The method may not include administering a priming dose of the recombinant fusion protein or dose ramp-up administrations of the recombinant fusion protein to mitigate on-target anemia.

The method may produce more than 85%, 90% or 95% of objective response rate after 16 weeks or longer of the treating. The objective response rate may be defined as the sum of complete response rate, marrow complete response rate, marrow complete response with hematological improvement rate and hematological improvement rate.

The method may produce more than 30%, 35%, 40%, or 45% of complete response rate after 16 weeks or longer of the treating.

In the method, no more than 5% or 10% of subjects receiving the administering of step (a) and the repeated administering of step (c) have chance of treatment-related hemolytic anemia.

In the method, not more than 5% or 10% of subjects receiving the of step (a) and the repeated administering of step (c) have chance of treatment-related grade 3 and 4 hemolysis.

In the method, not more than 15% or 20% of subjects receiving the of step (a) and the repeated administering of step (c) have chance of treatment discontinuation due to treatment-related adverse effects.

In a third aspect, the disclosure may provide a method of treating myelodysplastic syndrome in a subject in need thereof, which may comprise the steps of:
(a) administering intravenously to the subject for the myelodysplastic syndrome about 2.0 mg/kg body weight per day of a recombinant fusion protein in the form of a composition comprising a pharmaceutically acceptable excipient and the recombinant fusion protein,
(b) administering subcutaneously to the subject about 75 mg/m² of azacitidine, and
(c) after steps (a) and (b), repeating step (a) once weekly, and administering subcutaneously to the subject about 75 mg/m² of azacitidine once daily.

The step (b) may be performed about 65 minutes to about 75 minutes after completing the administering of step (a).

In step (c), the azacitidine is administered about 65 minutes to about 75 minutes after completing the repeated administering of step (a) on the days that the subject is also having step (a) repeated.

The recombinant fusion protein may comprise a mutated SIRPαD1 and a functional IgG1 heavy chain constant region. The mutated SIRPαD1 may be human SIRPαD1 comprising an asparagine (Asn, N) to alanine (Ala, A) mutation at a site corresponding to the position 80 of SEQ ID NO: 2. The mutated SIRPαD1 may comprise the amino acid sequence of SEQ ID NO: 2. In certain embodiments, the mutated SIRPαD1 may consist of the amino acid sequence of SEQ ID NO: 2. The functional IgG1 heavy chain constant region may be an IgG1 heavy chain constant region or a fragment thereof (e.g., a Fc region) that is able to bind a Fc receptor to induce recombinant fusion protein dependent cell mediated cytotoxicity or bind a complement system protein to induce complement dependent cytotoxicity. The functional IgG1 heavy chain constant region may be human IgG1 heavy chain constant region, or the Fc region thereof. The functional IgG1 heavy chain constant region may be the Fc region of human IgG1 heavy chain constant region, comprising e.g., the amino acid sequence of SEQ ID NO: 3. The functional IgG1 heavy chain constant region may, in certain embodiments, consist of the amino acid sequence of SEQ ID NO: 3. The recombinant fusion protein may comprise, from the N terminus to the C terminus, the mutated SIRPαD1 and the functional IgG1 heavy chain constant region. The recombinant fusion protein may comprise the amino acid sequence of SEQ ID NO: 1, and may consist of the amino acid sequence of SEQ ID NO: 1 in certain embodiments.

The method may not include administering a priming dose of the recombinant fusion protein or dose ramp-up administrations of the recombinant fusion protein to mitigate on-target anemia. Specifically, the administering of step (a) and the repeated administering of step (a) do not include administering a priming dose of the recombinant fusion protein or dose ramp-up administrations of the recombinant fusion protein to mitigate on-target anemia.

The method may produce more than 70%, 75% or 80% of objective response rate. The objective response rate may be defined as the sum of complete response rate, marrow complete response rate, marrow complete response with hematological improvement rate and hematological improvement rate.

The method may produce more than 20% or 25% of complete response rate after 16 weeks or longer of the treating.

In the method, no more than 5% or 10% of subjects receiving the administering of step (a) and the repeated administering of step (c) have chance of treatment-related hemolytic anemia.

In the method, not more than 5% or 10% of subjects receiving the of step (a) and the repeated administering of step (c) have chance of treatment-related grade 3 and 4 hemolysis.

In the method, not more than 5%, 10%, 15% or 20% of subjects receiving the of step (a) and the repeated administering of step (c) have chance of treatment discontinuation due to treatment-related adverse effects.

Other features and advantages of the instant disclosure will be apparent from the following detailed description and examples, which should not be construed as limiting. The contents of all references, Genbank entries, patents and published patent applications cited throughout this application are expressly incorporated herein by reference.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. § 112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. All rights to explicitly disclaim any embodiments that are the subject of any granted patent(s) of applicant in the lineage of this application or in any other lineage or in any prior filed application of any third party is explicitly reserved. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

### DESCRIPTION OF THE DRAWINGS

The following detailed description, given by way of example, but not intended to limit the invention solely to the specific embodiments described, may best be understood in conjunction with the accompanying drawings.

FIG. 1 shows the binding capability of IMM01 to red blood cells.

FIG. 2 shows the response rates, including objective response rate (ORR) and complete response (CR), of CMML patients treated with IMM01 and azacitidine (AZA) combination in comparison to several major clinical studies.

FIG. 3 shows the CMML patients' individual best percentage change in bone marrow blast from baseline, #represents >100% change from baseline, ⋆ represents less than 5% change from baseline.

FIG. 4 shows the CMML patients' individual response and duration of response.

FIG. 5 shows the MDS patients' individual best percentage change in bone marrow blast from baseline, #represents >100% change from baseline, ⋆ represents less than 5% change from baseline.

FIG. 6 shows the CMML patients' individual response and duration of response.

### DETAILED DESCRIPTION OF THE INVENTION

Myelodysplastic syndrome (MDS) is a kind of cancer that starts in the bone marrow, while chronic myelomonocytic leukemia (CMML) is a type of cancer that begins in the blood-forming cells in the bone marrow.

The cancer cells may have developed several ways to escape from host immune surveillance. For example, the cancer cells may express on surfaces a high level of CD47 proteins which may bind to the signal regulatory protein alpha (SIRPα) on macrophage surfaces, thereby inducing inhibitory signals that inhibit the phagocytosis of cancer cells by macrophages.

The signal regulatory protein (SIRP) is a trans-membrane glycoprotein, including three family members, SIRPα (CD172a), SIRPβ (CD172b) and SIRPγ (CD172g). All three proteins comprise similar extracellular regions but distinct intracellular domains. The extracellular region contains three immunoglobulin-like domains, one Ig V-set and two Ig C-set domains. The intracellular domain of SIRPα contains two inhibitory signaling regions that can inhibit signal transduction and corresponding cell functions. SIRPβ and SIRPγ have very short intracellular regions without any signal transduction domain. However, SIRPβ may function through an adaptor protein, e.g., DAP12 for signal transduction. SIRPs are mainly expressed on macrophages (Mφ), dendritic cells (DCs) and neurons.

CD47 is a transmembrane glycoprotein belonging to the immunoglobulin superfamily, and is expressed on the surface of all cell types including red blood cells. Ligands for CD47 include integrins, thrombospondin-1 and SIRPs. CD47, by interacting with SIRPα to emit a ‘don't eat me’ signal, can inhibit phagocytosis by macrophages and thus protects cells, such as blood cells, from being attacked by macrophages.

Studies have shown that many tumor or cancer cells over-express CD47s, which prevent phagocytosis of the cancer cells by macrophages. Cancer cells that over-express CD47 include cells of acute myeloid leukemia (AML), chronic myeloid leukemia (CML), acute lymphoblastic leukemia (ALL), non-Hodgkin's lymphoma (NHL), multiple myeloma (MM), bladder cancer, ovarian cancer, prostate cancer, lung cancer, colon cancer, breast cancer, and pancreatic cancer. It is reported that injection of CD47 specific antibody that blocks the binding of CD47 to SIRPα can significantly inhibit tumor growth in tumor-bearing mice. Tumor or cancer cells were eliminated completely when the same antibody was injected into the mice carrying human leukemia cells (Theocharides, A.P.A. et al., (2012) J. Exp. Med. 209(10): 1883-1899).

Conventional anti-CD47 antibodies bind intensely to CD47s on normal human red blood cell and thus produce an “antigen sink” effect, revealing comparatively less effect on tumor cells and requiring much higher dose (at 10-30 mg/kg) for efficacy. Additionally, to avoid hemolysis and anemia caused by CD47 binding on red blood cells, conventional anti-CD47 antibodies adopt an IgG4 or IgG2 design in their Fc region and may even require priming or “ramp-up” dose, as is the case for magrolimab. Consequentially, anti-CD47 antibodies reveal high hematological toxicity and minimal single-agent efficacy, and require combination therapy (with another tumor killing agent) to achieve in vivo anti-tumor efficacy (Joseph Maakaron et al., (2022) Blood 140 (Supplement 1): 3728-3730; David Andrew Sallman et al., (2023) Journal of Clinical Oncology 40(no. 16_suppl): 7017-7017; Naval Guastad Daver. Journal of Clinical Oncology 40(no. 16_suppl): 7020-7020).

In contrast, the mutated SIRPαD1-Fc molecule of present application, termed as IMM01 (timdarpacept) or recombinant fusion protein of the disclosure, utilizes an SIRPα extracellular domain 1 as ligand trap for CD47 neutralization, which is more tumor-specific than anti-CD47 antibodies, and reveals minimal binding to red blood cell in vitro (see Example 1).The SIRPαD1 of IMM01 contains a single asparagine to alanine point mutation at position 80 of the SIRPα sequence. As illustrated in FIG. 7B of U.S. Pat. No. 10,800,821B2, HY03M, an otherwise identical sequence having nine more amino acids at amino terminal than IMM01, binds to CD47 proteins on Jurkat leukemia cells with higher affinity than the wild type SIRPαD1-Fc molecule does. Due to its high tumor specificity, IMM01 also adopts an IgG1 design in its Fc portion for significantly more effective single-agent anti-tumor efficacy.In fact, FIG. 10B of U.S. Pat. No. 10,800,821B2 shows that HY03MM, an IgG1 defective version of HY03M, has much less anti-tumor efficacy than the IgG1 competent HY03M version. The data in the present application also show that IMM01 (timdarpacept) requires no priming dose and no more than 2.0 mg/kg to produce single agent in vivo efficacy, produces minimal hemolytic anemia, minimal grade 3 and 4 treatment-related hemolysis or treatment discontinuation due to treatment-related adverse reactions (Examples 2 to 6).

In addition to IMM01 (timdarpacept), Trillium TTI-621, TTI-622 and ALX Oncology's evorpacept (ALX148) also adopt a SIRPα-Fc molecular design, though the four sequences differ from one another.

Specifically, IMM01 (timdarpacept) employs 133 amino acids from the SIRPα extracellular domain 1 with a single (asparagine to alanine) point mutation at position 80, and an IgG1 Fc. TTI-621 employs 118 amino acids from the wild type SIRPα extracellular domain 1 and an IgG1 Fc, while TTI-622 employs the same 118 amino acids from the wild type SIRPα domain 1 and an IgG4 Fc. TTI-621 dose of up to 2.0 mg/kg is being tested in phase Ib/II trials for lymphoma and leiomyosarcoma treatment, while TTI-622 dose of around 8.0 mg/kg is being tested in phase Ib/II trials for treatment of lymphoma, acute myeloid leukemia (AML), multiple myeloma (MM) and other solid tumors. ALX48 uses a high affinity 118 amino acid SIRPα sequence with nine point mutations, which does not include present IMM01's N80A mutation, along with an inert IgG1 Fc. ALX148 binds to human red blood cells in vitro. Dose of more than 10.0 mg/kg of ALX148 are being tested in phase Ib/II trials for Non-Hodgkin lymphoma (NHL), myelodysplastic syndromes (MDS), AML, gastric cancer and head and neck squamous cell carcinoma (HNSCC).

The mutated SIRPαD1-Fc molecule of present application is used in a combination therapy for CCML or MDS with azacitidine.

The mutated SIRPαD1-Fc molecule of present application comprises i) a mutated SIRPαD1, as a ligand trap for CD47 neutralization, and ii) a functional IgG1heavy chain constant region such as the Fc region, which may induce recombinant fusion protein dependent cell mediated cytotoxicity or complement dependent cytotoxicity against cells bound by the mutated SIRPαD1. An example of the mutated SIRPαD1-Fc molecule is IMM01. Two copies of the mutated SIRPαD1-Fc molecule may dimerize to form an IgG1 antibody-like molecule through disulfide bonds between the functional IgG1 heavy chain constant regions. As an IgG1 antibody-like molecule, the dimer of the mutated SIRPαD1-Fc molecule has the general structure of an IgG1 antibody, with the SIRPαD1 part being the “paratope” or CD47-binding portion.

The mutated SIRPαD1 may be human SIRPαD1 comprising an asparagine (Asn, N) to alanine (Ala, A) mutation at a site corresponding to the position 80 of SEQ ID NO: 2. The mutated SIRPαD1 may comprise the amino acid sequence of SEQ ID NO: 2. In certain embodiments, the mutated SIRPαD1 may consist of the amino acid sequence of SEQ ID NO: 2.

The “functional IgG1 heavy chain constant region” refers to an IgG1 heavy chain constant region or a fragment thereof (e.g., a Fc region) that is able to bind a Fc receptor to induce recombinant fusion protein dependent cell mediated cytotoxicity or bind a complement system protein to induce complement dependent cytotoxicity. The functional IgG1 heavy chain constant region needs to contain the Fc region to exert said cytotoxicity.

The Fc region, i.e., the fragment crystallizable region, is the tail region of an antibody and is the domain that determines the effector function of the antibody, that is, how it engages with specific cell receptors or other defense proteins. An Fc region may interact with Fc receptors and/or proteins of the complement system, activating the immune system. For example, Fc receptors may bind to Fc-containing molecules (e.g., the antibodies, or the recombinant fusion proteins of the disclosure) that are attached to infected cells or invading pathogens, stimulating phagocytic or cytotoxic cells to destroy microbes or infected cells. Fc receptors (FcRs) are found on the surface of certain immune effector cells, including B lymphocytes, follicμlar dendritic cells, natural killer cells, macrophages, neutrophils, eosinophils, basophils, and mast cells.

The term “recombinant fusion protein dependent cell mediated cytotoxicity” herein refers to an immune mechanism through which the Fc receptor-bearing effector cells recognize and kill Fc-containing molecule (e.g., the recombinant fusion protein of the disclosure such as IMM01)-coated target cells.

The “complement dependent cytotoxicity” or “CDC” is a mechanism by which a Fc-containing molecule (e.g., the recombinant fusion protein of the disclosure such as IMM01) mediates specific target cell lysis through activation of an organism's complement system. CDC is initiated when C1q, the initiating component of the classical complement pathway, is fixed to the Fc portion of target cell-bound Fc-containing molecules.

In certain embodiments, the functional IgG1 heavy chain constant region may be the Fc region of human IgG1 heavy chain constant region, comprising e.g., the amino acid sequence of SEQ ID NO: 3. The functional IgG1 heavy chain constant region may, in certain embodiments, consist of the amino acid sequence of SEQ ID NO: 3.

The mutated SIRPαD1-Fc molecule may comprise, from the N terminus to the C terminus, the mutated SIRPαD1 and the functional IgG1 heavy chain constant region. The mutated SIRPαD1-Fc molecule may comprise the amino acid sequence of SEQ ID NO: 1, and may consist of the amino acid sequence of SEQ ID NO: 1 in certain embodiments.

The disclosure may provide a composition, e.g., a pharmaceutical composition, that may comprise the mutated SIRPαD1-Fc molecule and azacitidine. The mutated SIRPαD1-Fc molecule may be formulated together with a pharmaceutically acceptable carrier. Azacitidine may be formulated together with a pharmaceutically acceptable carrier.

A pharmaceutically acceptable carrier means a carrier that is useful in preparing a pharmaceutical composition that is generally safe, non-toxic and neither biologically nor otherwise undesirable, and includes a carrier that is acceptable for veterinary use as well as human pharmaceutical use. The primary carrier used in a pharmaceutical composition may be either aqueous or non-aqueous in nature. For example, a suitable carrier may be water for injection, physiological saline solution or artificial cerebrospinal fluid, possibly supplemented with other materials common in injection. For example, the vehicle or carrier may be neutral buffered saline or saline mixed with serum albumin. Other exemplary pharmaceutical compositions comprise Tris buffers, or acetate buffers, which may further include sorbitol or a suitable substitute thereof. In one embodiment of the present disclosure, the compositions may be prepared for storage by mixing the selected mutated SIRPαD1-Fc molecule or azacitidine having the desired degree of purity with optional formulation agents in the form of a lyophilized cake or an aqueous solution. Further, the composition may be formulated as a lyophilizate using appropriate excipients such as sucrose.

The pharmaceutical composition can comprise any number of excipients. Excipients that can be used include carriers, surface active agents, thickening or emulsifying agents, binders, dispersion or suspension aids, solubilizers, colorants, flavoring agents, coatings, disintegrating agents, lubricants, sweeteners, preservatives, isotonic agents, and combinations thereof. The selection and use of suitable excipients are taught in Gennaro, ed., Remington: The Science and Practice of Pharmacy, 20th Ed. (Lippincott Williams & Wilkins 2003).

The mutated SIRPαD1-Fc molecule and azacitidine may be formulated for intravenous, intramuscular, subcutaneous, parenteral, spinal or epidermal administration (e.g., by injection or infusion). Depending on the route of administration, the mutated SIRPαD1-Fc molecule or azacitidine can be coated in a material to protect it from the action of acids and other natural conditions that may inactivate it. The phrase “parenteral administration” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion. Particularly, the mutated SIRPαD1-Fc molecule may be intravenously administered, and azacitidine may be subcutaneously administered.

The amount of the recombinant fusion protein or azacitidine which can be combined with a carrier material to produce a single dosage form will vary depending upon the subject being treated, the particular mode of administration, the lesion site, etc. and will generally be that amount which produces a therapeutic effect.

Particularly, the mutated SIRPαD1-Fc molecule of the disclosure may be provided in the form of a protein dispersion, or lyophilized power which can be dispersed in e.g., water for injection, whatever applicable. In certain embodiments, IMM01 in 0.9% physiological saline solution (or normal saline) is used for administration in the disclosure.

Particularly, azacitidine may be provided in the form of a suspension for injection, suitable for subcutaneous use, or tablets for oral administration. In certain embodiments, azacitidine should be in a form applicable to subcutaneous use. In certain embodiments, azacitidine (VIDAZA®, Bristol myers squibb) is used for administration in the disclosure.

Dosage regimens may be adjusted to provide the optimum desired response (e.g., a therapeutic response). For example, several divided doses can be administered over time or the dose can be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active molecule calculated to produce the desired therapeutic effect in association with the required pharmaceutical carrier. Alternatively, the mutated SIRPαD1-Fc molecule or azactidine can be administered as a sustained release formulation, in which case less frequent administration is required.

In another aspect, the present application provides a method for treating chronic myelomonocytic leukemia (CMML) or myelodysplastic syndrome (MDS) in a subject in need thereof.

The clinical trial design for the treatment method of the disclosure is as follows.

The traditional 3+3 design is used for dose escalation and determination of maximum tolerated dose (MTD) and recommended phase 2 dose (RP2D) of the IMM01+azacutudube (AZA) combination in phase I clinical trial.

According to the phase I clinical trial design, the IMM01 protein is administrated via intravenous infusion at the dose of 1.0 mg/kg, 1.5 mg/kg or 2.0 mg/kg, once a week, 28 days as a treatment cycle, up to 12 treatment cycles, and azacitidine is administered subcutaneously at a fixed dose of 75.0 mg/m²/day for 7 consecutive days in a 28-day cycle, up to 12 treatment cycles. The administration is stopped when a patient experiences intolerable toxicity, disease progression, loss to follow-up, death, withdrawal of consent, discontinuation of treatment in the best interest of the patient in the opinion of the investigator, receipt of other anti-tumor therapy, 48 weeks of treatment, or early termination of the study, whichever occurs first.

Bone marrow cytology is performed before Day 1 of Cycle 2 (time window of —7 days), and efficacy assessment during the treatment period does not affect the subjects' continuation of treatment. Patients with MDS or CMML are examined and assessed every treatment cycle if they do not achieve complete response (CR), or marrow complete response (mCR) with hematological improvement (HI); patients are double checked before the next treatment cycle and assessed every 2 treatment cycles (8 weeks [window: −7 days]) thereafter, if CR or mCR+HI is achieved; and patients are examined and assessed every treatment cycle or at the investigator's discretion if mCR is achieved. Patients are closely observed for adverse events from the start of the first dose until 28 days after the last dose (window: ±7 days). Care is taken to avoid the use of granulocyte colony-stimulating factor, blood transfusion or other behaviors that may affect the results during efficacy evaluation.

The phase I trial should be ended upon occurrence of the last subject withdrawing consent, terminating treatment or withdrawing from the trial, lost to follow-up or death, being treated for 48 weeks or ending the study prematurely, whichever occurs first.

MTD is defined as the maximum dose at which the incidence of dose-limiting toxicity (DLT) is <⅓. For the dose level to be determined as MTD, there must be at least 6 subjects with evaluable DLT data. Once MTD is determined, MTD is usually used as RP2D, or the dose level below MTD can be selected as RP2D.

RP2D is initially determined based on the safety, PK, efficacy, and other data of dose escalation in the combination therapy, in combination with the safety, tolerability and PK/PD data of the dose escalation and dose expansion in IMM01 monotherapy, and finally decided by the scientist review committee (SRC).

For phase II clinical study, which is non-randomized with parallel assignment model, without masking, IMM01 is intravenously administered once a week at the RP2D, with every 28 days as a treatment cycle, and azacitidine at 75.0 mg/m²/day is administered once daily subcutaneously, with each 28-day treatment as one treatment cycle. On days when both IMM01 and azacitidine are administered, the azacitidine administration is at about 65 minutes to about 75 minutes after the IMM01 injection.

For phase II clinical study, the inclusion criteria for previously untreated CMML cohorts and previously untreated MDS cohorts include:
1. The subjects should be able to voluntarily sign the informed consent form and communicate well with the investigator, and should be willing to comply with study-related regulations.
2. Male or female ≥18 years of age.
3. Eastern Cooperative Oncology Group (ECOG) score of 0 to 2.
4. Expected survival ≥12 weeks.
5. Upon signing the Informed Consent Form, females and males of childbearing potential must agree to practice effective contraception during the study and for 3 months after the last dose of IMM01, and females patients of childbearing potential must have a negative result for pregnancy test within 7 days prior to first dose.
6. White blood cell count ≤20×10⁹/L prior to the first dose of the study drugs (hydroxyurea treatment is allowed, but not within 3 days prior to the first dose of study drugs).
7. Consent for bone marrow aspirate and biopsy at screening and during treatment.
8. Appropriate organ function, defined as follows:
   8a. Serum creatinine ≤1.5 times upper limit of normal (MLN) or endogenous creatinine clearance (CrCl) ≥50 ml/min/1.73 m² as estimated by the Cockcroft-Gault formula;
   8b. Aspartate aminotransferase (AST) and alanine aminotransferase (ALT)≤2.5 times MLN;
   8c. Serum total bilirubin (TBIL)≤1.5 times MLN, and TBIL≤3 times MLN in patients with a history of Gilbert's syndrome;
   8d. International normalized ratio (INR) or prothrombin time (PT)≤1.5×MLN; activated partial thromboplastin time (aPTT)≤1.5×MLN; INR≤2×MLN if treated with warfarin anticoagulation.
9. MDS patients should reveal IPSS-R of over 3.5 (see Table 1, Wu Depei et al., (2019) Chinese Guidelines for the Diagnosis and Treatment of Myelodysplastic Syndrome (2019 Edition). Chinese Journal of Hematology 40 (2): 89-97).

10. CMML patient recruitment follows guideline of 2016 WHO (Table 2, Wu Depei et al., (2019) Supra) and CMML types I and II patients should be included.

TABLE 1
MDS Revised International Prognostic Scoring System (IPSS-R)

| | Score | | | | | | |
|---|---|---|---|---|---|---|---|
| | 0 | 0.5 | 1 | 1.5 | 2 | 3 | 4 |
| Cytogeneticsª | Excellent | | Good | | Moderate | Poor | Very poor |
| Bone marrow cells (%) | ≤2 | | >2~<5 | | 5~10 | >10 | |
| Hemoglobin (g/L) | ≥100 | | 80~≤100 | <80 | | | |
| Platelet count (×10⁹) | ≥100 | 50~≤100 | <50 | | | | |
| Absolute neutrophil | ≥0.8 | <0.8 | | | | | |
| count (×10⁹) | | | | | | | |

Note:
ªExcellent: −Y, del (11q); good: normal karyotype, del (5q), 12 p−, del (20q), del (5q) plus another abnormality; moderate: del (7q), +8, +19, i (17q), chromosomal abnormalities in one or two other independent clones; poor: −7, inv (3)/t (3q)/del (3q), −7/del (7q) plus another abnormality, complex abnormality (3); very poor: complex abnormality (>3).
IPSS-R risk classification: very low risk: ≤1.5 points; low risk: >1.5-3 points; intermediate risk: >3-4.5 points; high risk: >4.5-6 points; very high risk: >6 points.

TABLE 2
WHO (2016) Myelodysplastic Syndrome (MDS) Revised Classification

| | | Blood | | Bone marrow | |
|---|---|---|---|---|---|
| | | cells | Annular | and peripheral | Routine |
| Disease type | Dysplasia | decreased | sideroblasts | blood blasts | karyotyping |
| MDS with | 1 | 1-2 | <15% or | Bone marrow <5%, | Any karyotype, but |
| monophyletic | | | <5% ^{a} | peripheral blood | not meeting |
| blood cell | | | | <1%, no Auer bodies | criteria for MDS |
| dysplasia (MDS-SLD) | | | | | with pure del (5q) |
| MDS with | 2-3 | 1-3 | <15% or | Bone marrow <5%, | Any karyotype, but |
| multilineage | | | <5% ª | peripheral blood | not meeting |
| dysplasia (MDS-MLD) | | | | <1%, no Auer bodies | criteria for MDS |
| | | | | | with pure del (5q) |

MDS with ring sideroblasts (MDS-RS)

| | | | | | |
|---|---|---|---|---|---|
| MDS-RS-SLD | 1 | 1-2 | ≥15% or | Bone marrow <5%, | Any karyotype, but |
| | | | ≥5% ^{a} | peripheral blood | not meeting |
| | | | | <1%, no Auer bodies | criteria for MDS |
| | | | | | with pure del (5q) |
| MDS-RS-MLD | 2-3 | 1-3 | ≥15% or | Bone marrow <5%, | Any karyotype, but |
| | | | ≥5% ^{a} | peripheral blood | not meeting |
| | | | | <1%, no Auer bodies | criteria for MDS |
| | | | | | with pure del (5q) |
| MDS with pure del | 1-3 | 1-2 | Any ratio | Bone marrow <5%, | Del (5q) only, can |
| (5q) | | | | peripheral blood | be associated with |
| | | | | <1%, no Auer bodies | 1 other abnormal |
| | | | | | [except −7 or del |
| | | | | | (7q)] |

MDS with excess blasts (MDS-EB)

| | | | | | |
|---|---|---|---|---|---|
| MDS-EB-1 | 0-3 | 1-3 | Any ratio | 5% to 9% in bone | Any karyotype |
| | | | | marrow or 2% to 4% | |
| | | | | in peripheral blood, | |
| | | | | no Auer bodies | |
| MDS-EB-2 | 0-3 | 1-3 | Any ratio | 10% to 19% in bone | Any karyotype |
| | | | | marrow or 5% to | |
| | | | | 19% in peripheral | |
| | | | | blood or Auer bodies | |

MDS, not classifiable (MDS-U)

| | | | | | |
|---|---|---|---|---|---|
| Peripheral blood | 1-3 | 1-3 | Any ratio | Bone marrow <5%, | Any karyotype |
| blasts 1% | | | | peripheral blood = | |
| | | | | 1% ^{b}, no Auer bodies | |
| Unilineage blood | 1 | 3 | Any ratio | Bone marrow <5%, | Any karyotype |
| cell dysplasia with | | | | peripheral blood | |
| pancytopenia | | | | <1%, no Auer bodies | |
| With diagnostic | 0 | 1-3 | <1.5% ^{c} | Bone marrow <5%, | Abnormal |
| abnormal | | | | peripheral blood | karyotype with |
| karyotype | | | | <1%, no Auer bodies | defined MDS |

Note:
MDS: myelodysplastic syndrome;
cytopenias are defined as hemoglobin <100 g/L, platelet count <100 × 10⁹/L, absolute neutrophil count <1.8 × 10⁹/L, and in rare cases mild anemia or hrombocytopenia above these levels is seen in MDS, and peripheral blood mononuclear cells must be <1 × 10⁹/L;
ª if SF3B1 mutations are present;
^{b} blasts in peripheral blood = 1% must be documented on two separate occasions;
^{c} if ring sideroblasts are ≥15% of cases with marked erythroid dysplasia, they are classified as MDS-RS-SLD.

The exclusion criteria include:
1. Have received previous treatment with an anti-CD47 monoclonal antibody or an SIRPα fusion protein.
2. Have received allogeneic hematopoietic stem cell transplantation or other organ transplantation, or had autologous hematopoietic stem cell transplantation previously within less than 6 months.
3. Uncorrected red cell folate deficiency or myelodysplastic syndrome with vitamin B12 deficiency (may be re-screened after treatment with vitamin B12 and folic acid).
4. Other malignant tumors within 5 years prior to enrollment, with the following exceptions:
   4a. Cured cervical carcinoma in situ or non-melanoma skin cancer;
   4b. Patients who had complete disease remission for at least 2 years before the first dose and who do not require anti-tumor therapy.
5. Patients with a history of active autoimmune diseases, including but not limited to systemic lupus erythematosus, psoriasis, rheumatoid arthritis, inflammatory bowel disease, Hashimoto's thyroiditis, autoimmune thyroid disease, multiple sclerosis, etc., with except for the following diseases:
   5a. Hypothyroidism that can be controlled by hormone replacement therapy alone;
   5b. Dermatosis that does not require systemic therapies (such as vitiligo, psoriasis);
   5c. Type I diabetes requiring insulin therapy only.
6. Patients who had treatment with major surgery within 4 weeks prior to the first dose, or minor surgical procedures (including catheterization, excluding bone puncture and peripherally inserted central catheter) within 2 days before screening.
7. Subjects who require systemic corticosteroids (equivalent to >10 mg prednisone/day) or other immunosuppressive agents within 14 days prior to the first dose or during the study, with enrollment allowed in the following circumstances:
   7a. The subjects are permitted to use topical or inhaled glucocorticoids;
   7b. Short-term (≤7 days) use of glucocorticoids is permitted for the prevention or treatment of non-autoimmune allergic diseases.
8. Hypertension (systolic blood pressure ≥140 mmHg or diastolic blood pressure ≥90 mmHg), pulmonary hypertension, or unstable angina, which cannot be controlled by medical treatment; treatment with myocardial infarction, bypass or stent surgery within 6 months prior to first dose; a history of chronic heart failure rated by the New York Heart Association (NYHA) as Grade 3-4; valvular disease with clinical significance; severe arrhythmia requiring treatment (excluding atrial fibrillation and paroxysmal supraventricular tachycardia), including QTc interval ≥450 ms for males and ≥470 ms for females (calculated by Fridericia formula); cerebrovascular accident (CVA) or transient ischemic attack (TIA) within 12 months prior to first dose.
9. Patients with a history of arterial thrombosis or deep venous thrombosis within 6 months prior to enrollment, or patients with a history of spontaneous cerebral hemorrhage within 2 months prior to enrollment, regardless of severity.
10. Severe gastrointestinal diseases, such as duodenal ulcer, esophageal varices, intestinal obstruction, acute Crohn's disease, ulcerative colitis, massive gastric and small intestinal resection, etc.; previous history of intestinal perforation, intestinal fistula disease, but not recovered after surgical treatment.
11. Patients with active lung disease, interstitial lung disease or pneumonia (except local interstitial pneumonia induced by radiotherapy), pulmonary fibrosis, severe dyspnea, pulmonary insufficiency or continuous oxygen inhalation.
12. Definite evidence of active infection prior to enrollment, with for example, intravenous antibiotic infusion and normothermia for less than 72 hours, no improvement in the original infected lesion or the appearance of new lesions as measured by imaging, or the presence of unhealed wounds.
13. HBsAg positive or HBcAb positive, except those, after receiving anti-hepatitis B virus treatment, are not in the active phase in the opinion of the investigator with the retest of HBV DNA quantification lower than the upper limit of normal value and able to receive continuous antiviral therapy during the trial (retest of hepatitis B virus antigen/antibody and HBV-DNA performed at end of each efficacy evaluation cycle); positive HCV-RNA qualitative results or quantitative results>the upper limit of normal; human immunodeficiency virus (HIV) antibody positive.
14. Have received live attenuated vaccine within 4 weeks before the first dose.
15. Patients with a previous history of severe hypersensitivity to protein drugs (CTCAE v5.0>Grade 3); or patients with hypersensitivity to azacitidine.
16. Participated in other drug clinical trials 28 days prior to the first dose.
17. History of neurological or psychiatric disorders, such as epilepsy, dementia, or alcohol abuse or drug abuse that will affect compliance.
18. Other situations where the patients are inappropriate for participation in this clinical trial in the opinion of the investigator.

The patients meeting any of the criteria above cannot be enrolled in this study.

The primary endpoints for phase II include:
Incidence and characteristics of adverse events, and changes from baseline in laboratory test results, physical examinations, electrocardiograms, and vital signs;
Dose-Limiting Toxicity (DLT), Incidence rate and the grade (severity) of DLTs;
Maximum Tolerated Dose (MTD), which is the highest dose in patients with DLT incidence <⅓; for a dose group to be assessed as MTD, at least 6 DLT data must be available to evaluate the subject.

The secondary endpoints for phase II include Pharmacokinetic (PK) evaluation indicators of IMM01, including, but not limited to:
Cmax—maximum observed concentration in serum within 60 minutes prior to administration on Day 1, Day 8, and Day 15 of Cycle 1 and Day 1 of Cycle 2 to 6, 10 minutes post-administration on Day 15 of Cycle 1 and Day 22 of Cycle 6, and 10 minutes and 4 hours post-administration on Day 1 of Cycle 1.
Area under the plasma concentration-time curve over the dosing interval (AUC_{0-τ}), mean steady-state plasma concentration (C_{avg}), minimum plasma concentration (Cₘᵢₙ), steady-state clearance (CLₛₛ), time to peak (maximum) serum concentration, terminal half-life (T_{1/2}), etc.

Treatment efficacy evaluation of MDS and CMML follows the International Working Group (IWG) response criteria in myelodysplasia published in 2006 (Table 3, Cheson BD et al., (2006) Clinical application and proposal for modification of the International Working Group (IWG) response criteria in myelodysplasia. Blood. 108(2): 419-425), and the efficacy evaluation indicators include:

1. MDS/CMML efficacy, which will be assessed using the International Working Group (IWG) response criteria for myelodysplastic syndrome, including:
Complete remission (CR),
Partial remission (PR),
Bone marrow complete remission (marrow CR),
Stable disease (SD),
Hematologic improvement (efficacy must be maintained for ≥8 weeks), including erythroid response, platelet response, and neutrophil response,
Cytogenetic response,
Progressive disease (PD),
Survival, including event-free survival, progression-free survival, disease-free survival, etc.

2. Overall survival (OS, the number of days from enrollment to death).

Exploratory endpoints and immunogenicity indicators include:
Anti-drug antibody (ADA) and neutralizing antibody [Nab] (only for subjects with positive ADA);
Correlation between biomarkers and efficacy.

TABLE 3
International Working Group (IWG) Response Criteria for Myelodysplastic
Syndrome (IWG 2006 Revision)

| | |
|---|---|
| Category | Criteria for efficacy (efficacy must be maintained |
| | for ≥4 weeks) |
| Complete Remission | Bone marrow: ≤5% blasts and all cell lines are mature |
| | and normal ^{a} |
| | Persistent hemocyte dysplasiaa should be noted ^{a} |
| | Peripheral blood: HGB ≥110 g/L, ANC ≥1.0 × 10⁹/L, |
| | PLT ≥100 × 10⁹/L, blasts: 0 |
| Partial remission | Absolute peripheral blood must have persisted for at |
| | least 2 months |
| | Other conditions met the criteria for CR (where there |
| | isan abnormality before treatment), but bone marrow |
| | blasts are only reduced by ≥50% compared with those |
| | before treatment, but still >5% regardless of the |
| | degree of bone marrow cell proliferation and |
| | morphology |
| Complete remission of bone marrow | Bone marrow: ≤5% blasts and ≥50% reduction from |
| | that before treatment |
| | Peripheral blood: also specify if hematologic |
| | improvement (HI) is achieved |
| Stable Disease | No minimum criteria for PR but no evidence of |
| | disease progression for at least 8 weeks |
| Hematologic improvement (efficacy | |
| must be maintained for ≥8 weeks) | |
| Erythroid reaction | HGB increased ≥15 g/L |
| (HGB before treatment <110 g/L) | |
| Platelet response (PLT before | PLT before treatment >20 × 10⁹/L, net increase ≥30 × |
| treatment <100 × 10⁹/L) | 10⁹/L or from <20 × 10⁹/L to >20 × 10⁹/L and at |
| | least 100% increase |
| Neutrophil response (ANC | More than 100% increase and absolute increase >0.5 × |
| before treatment <1.0 × 10⁹/L) | 10⁹\hat{ }/L |
| Treatment failure | Death or disease progression on treatment manifested |
| | by worsening cytopenias, increased bone marrow |
| Category | Criteria for efficacy (efficacy must be maintained |
| | for ≥4 weeks) |
| | blasts, or development of more progressive FAB |
| | subtypes than before treatment |
| Relapse following complete or partial | At least 1 of the following: |
| remission | Bone marrow blasts returned to pre-treatment levels |
| | 50% or more decrease in ANC or PLT from best |
| | overall response |
| | HGB decrease ≥15 g/L or transfusion-dependent ^{b} |
| Progression or relapse following | At least 1 of the following: |
| hematologic improvement ^{a} | ≥50% decrease in ANC or PLT from best overall |
| | response |
| | HGB decrease ≥15 g/L |
| | Transfusion-dependent ^{b} |
| Cytogenetic response | Complete remission: disappearance of chromosomal |
| | abnormalities and no new abnormalities |
| | Partial remission: ≥50% reduction in the proportion |
| | of cells with chromosomal abnormalities |
| Disease Progression | Blasts <5%: ≥50% increase in blasts to 5% |
| | 5% to 10% blasts: ≥50% increase in blasts to 10% |
| | 10% to 20% blasts: ≥50% increase in blasts to 20% |
| | 20% to 30% blasts: ≥50% increase in blasts to 30% |
| | Any of the following: |
| | ≥50% decrease in ANC or PLT from best overall |
| | response |
| | HGB decrease ≥20 g/L |
| | Transfusion-dependent ^{b} |
| Survival rate | End time: |
| | Overall survival: death from any cause |
| | Event-free survival: treatment failure or death from |
| | any cause |
| | Progression-free survival: disease progression or |
| | death due to MDS |
| | Disease-free survival: until relapse |
| | Death due to special cause: MDS-related death |

Note:
ª In the absence of other explanations such as infection, repeated chemotherapy courses, gastrointestinal bleeding, hemolysis, etc.
^{b} Transfusion of ≥2 units of red blood cells every 8 weeks

The safety and tolerability of IMM01+AZA are evaluated by monitoring serious adverse events, adverse events, physical examination results, vital signs, laboratory findings, 12-lead ECG, etc. from Day 1 of administration until 28 days after the last dose.

Adverse events occurring during the study are assessed according to National Cancer Institute Common Terminology Criteria for Adverse Events (NCI-CTCAE) version 5.0. Drug-related adverse events should be followed up until the parameter(s) return(s) to the baseline, the disease condition is stable in the opinion of the investigator, the patient initiates new treatment, the patient is lost to follow-up, the patient withdraws informed consent, or it is determined that the adverse event is not caused by the study drug(s).

The toxicity of the study drug(s) and the severity of the adverse events directly affect the dose escalation of the study drug. Dose-limiting toxicity (DLT) refers to toxicity observed within 1 to 28 days after the first dose that may be related to the study drug, unless it is confirmed that the toxicity is not related to IMM01 and/or azacytidine or there are other clear causes.

Non-hematologic toxicity includes:

1. ≥Grade 3 non-hematological toxicities, except:
Grade 3 nausea/vomiting, diarrhoea and/or electrolyte imbalance lasting <72 hours after best supportive treatment;
Grade 3 rash lasting <72 hours;
Grade 3 flu-like symptoms or fever, which can be controlled by medical supportive treatment;
Grade 3 fatigue lasting ≤2 weeks;
Adverse events ≥Grade 3 caused by disease progression, as determined by the investigator.

2. ≥Grade 3 serum creatinine (Cr>3.0×MLN) or ≥Grade 3 endogenous creatinine clearance (CrCl≤29 mL/min/1.73 m²).

3. Infusion-related reactions/allergic reactions.

The infusion-related reactions/allergic reactions are generally adverse reactions that often occur during intravenous infusion of protein drugs, and are not considered DLTs. However, if Grade ¾ infusion reactions are observed in a patient that the treatment should be disconnected, another patient will be added to this dose group. If more than 2 patients experience Grade ¾ infusion reactions at the same dose level, enrollment will be suspended, and the sponsor and investigator will discuss whether to use pretreatment and continue enrollment.

Hematologic toxicity includes: ≥Grade 4 hematologic toxicities lasting >4 weeks, including hematopoietic hypoplasia of the bone marrow (bone marrow test) lasting >4 weeks.

Grade 4 anemia, neutropenia, and thrombocytopenia are not considered DLTs if they are caused by the disease under study (AML or MDS) and are common symptoms of the disease under study. Grade 3 or higher asymptomatic laboratory abnormalities without clinical significance as judged by the investigator are not considered DLTs. In addition, serious toxicities observed in the study may be recorded as DLTs after discussion and confirmation by the investigator and the sponsor. Concomitant therapies include:

1. During the study, palliative and supportive treatments for symptoms associated with advanced tumors, including the use of colony-stimulating factors and blood transfusions and related blood products, are allowed, and drugs for adverse reactions are also permitted. Drugs that may induce or aggravate clinical study symptoms cannot be used, and symptom monitoring should be strengthened if medication of such drugs cannot be avoided. Phase IIa allows prophylactic administration to mitigate the nausea and vomiting toxicity of azacitidine, with the exception of corticosteroids.

2. Palliative local radiotherapy (within limited range) for pain relief is permitted, and bisphosphonates or denosumab are permitted if bone metastases have already occurred before enrollment. Subjects can use topical, ocular, intra-articular, intranasal, and inhaled corticosteroids. Systemic corticosteroids at physiologic replacement doses (i.e., prednisone ≤10 mg/day) are permitted. Short-term use of corticosteroids for prophylaxis (e.g., contrast media allergy), for non-autoimmune disorders (e.g., delayed hypersensitivity reactions due to contact allergens), or for management of adverse reactions caused by the investigational drug is permitted.

3. Oral contraceptives, hormone replacement therapy, prophylactic or therapeutic anticoagulants, and other permitted therapies may be continued. When using anticoagulants, investigators need to carefully assess the risk of bleeding in subjects. When using warfarin, pay attention to monitor the international normalized chemotherapy ratio INR ≤2 times MLN.

4. During the conduct of the clinical trial, if anti-HBV therapy is required, appropriate antiviral therapy will be administered at the investigator's discretion and appropriate HBV monitoring (including hepatitis B virus (HBV) 2 and a half pairs and HBV-DNA) will be performed.

The following treatments other than the investigational drugs are prohibited during the study.

1. In Phase 1b study period, drugs to prevent adverse reactions are not allowed before Cycle 1 except the prophylactic drugs which are allowed in the protocol, such as blood transfusion and drugs to prevent tumor lysis syndrome or drugs to prevent infusion-related reactions (IRRs).

2. During the DLT observation period, erythropoietin, megakaryocyte colony-stimulating factor or biological agents that can promote erythroid and megakaryocytic hyperplasia are not recommended, but granulocyte colony-stimulating factor is allowed but should be avoided as much as possible during the screening period, efficacy evaluation, and in the absence of disease remission.

3. Any concomitant anti-tumor treatments (chemotherapy, immunotherapy, biologic product, extensive radiation therapy, hormonal therapy, targeted therapy, surgery, interventional therapy, and device therapy), investigational therapy, or approved therapy are not allowed.

4. Immunosuppressants are not allowed, except for such drugs are used to treat immune-related adverse events.

5. Systemic corticosteroids with immunosuppressive effects are not allowed, except for temporary hormone replacement therapy for infusion reactions, immune-related adverse reactions, or adrenal insufficiency, in such cases, dosage of systemic corticosteroids for infusion reactions or immune-related adverse reactions shall be controlled at the daily dose of ≤10 mg prednisone or equivalent, and shall be reduced gradually before next dose. IMM01 administration should be temporarily discontinued if >10 mg prednisone is taken daily.

6. Concomitant medications (prescription, over-the-counter, or traditional Chinese medicines) are prohibited unless determined by the investigator as necessary to the subject for the treatment of a specific clinical event. Any Traditional Chinese Medicine approved for anti-cancer therapy is not allowed (i.e., anti-cancer or anti-tumor phrases in the Traditional Chinese Medicine package insert are not allowed). If needed, the investigator may decide to give non-anticancer therapy, supportive care of Traditional Chinese Medicine.

7. Immunization of live or attenuated vaccine is not allowed throughout the study.

Subjects are prematurely withdrawn from the study if any other specific anti-cancer treatment is required at the discretion of the investigator.

The sample sizes in statistical analysis are described as follows.

The phase Ib study used a 3+3 approach for dose escalation, with 3 to 6 patients in each dose group.

Phase IIa Cohorts 1-3 plan to enroll no more than 16 patients per cohort and

Cohort 4 plans to enroll no more than 90 patients (no more than 60 for MDS and no more than 30 for CMML).

The setting of the sample size is not based on statistical assumptions and the 90% CI for objective response rate (ORR) estimated by Clopper Pearson method is presented in the table 4 below. Simon's two-stage design is used to establish the statistical hypothesis for the CMML cohort, including H₀: ORR≤ 40% versus H₁≥80%, α=0.05 (one-sided), power 90%. If ≤3 responses are observed in 8 enrolled patients in the first stage, the study will be stopped. Otherwise, additional 5 patients will be enrolled (total sample size of 13). During the study, the actual number of patients recruited may be adjusted according to the SRC meeting resolution and actual situation.

TABLE 4
90% confidence interval for objective response rate

| Sample size (n) | Number of ORR | ORR % [90%CI] |
|---|---|---|
| 16 | 1 | 6.25% [0.32%, 26.40%] |
| | 2 | 12.5% [2.27%, 34.38%] |
| | 3 | 18.75% [5.31%, 41.66%] |
| | 4 | 25% [9.03%, 48.44%] |
| | 5 | 31.25% [13.21%, 54.83%] |
| | 6 | 37.5% [17.78%, 60.90%] |
| | 7 | 43.75% [22.67%, 66.66%] |
| | 8 | 50% [27.86%, 72.14%] |

The analysis population is described below.

The Safety Data Set (SAS) includes all subjects who have received at least one dose of the study drug(s), unless otherwise noted, and this data set is applicable to all analyses.

The Dose Determining Set (DDS) includes all subjects who have completed the minimum exposure requirement in the dose escalation phase and received sufficient safety assessment, or developed a DLT during the first treatment period of medication (28 days after the first dose).
Subjects are considered to have met the minimum exposure principle if they have completed at least 80% of the scheduled dose during the DLT observation period;
Subjects are considered to have received adequate safety assessments if they have had an observation period of more than 28 days after the first dose, even if no DLT occurs during the DLT observation period.

The Efficacy Data Set (EAS) includes all subjects who have received at least one dose of the study drug(s) and had at least one postbaseline efficacy evaluation. ORR and duration of response (DOR) are statistically analyzed using EAS.

The Pharmacokinetic Concentration Set (PKCS) includes all subjects who have received at least one dose of the study drug(s) and have at least one valid plasma concentration data for the tested component(s).

The Pharmacokinetic Parameter Analysis Set (PKPS) includes all subjects who have received at least one dose of the study drug(s) and have at least one valid pharmacokinetic parameter data.

The Immunogenicity Analysis Set (SAS) includes all subjects who have received at least one dose of the study drug(s) and have at least one post-dose immunogenicity sample available for analysis.

Statistical approaches: Descriptive statistics are used for summarization. In general, continuous variables (e.g., age) are statistically described using the value observed, mean, median, standard deviation, minimum, and maximum; categorical variables are statistically described using frequency and percentage. All variables obtained at each observation time point are statistically described by dose group. Quantitative or qualitative data are statistically analyzed using SAS (Version 9.4 or higher) statistical software.

Safety analysis: Data analysis set includes all enrolled patients who have received at least one dose of study drug. This data set is applicable to all analyses. The detailed statistical methods are specified in the statistical plan. All adverse events (including deaths and serious adverse events) are summarized by dose groups and tabulated by subjects. Adverse events are coded by organ system and standard terms using MedDRA, and adverse events are graded by NCI CTCAE v5.0. Inferential statistics are not required for safety indicators. Abnormal values of laboratory test findings, vital signs, ECG and other safety indicators should be indicated.

Efficacy analysis: Efficacy endpoints are analyzed using descriptive statistics. ORR is estimated using Clopper-pearson method, with corresponding 90% confidence interval provided. If the number of subjects is more than 10 in a dose group, PFS and DOR are analyzed using Kaplan-Meier method, with median and 90% confidence interval provided.

Cut-off date for primary data analysis: The scheduled cut-off date for primary data analysis is one year after enrollment of the last subject, or the date when the study is terminated prematurely, or until any of the following occurs (whichever occurs first): intolerable toxicity, disease progression, start of subsequent anti-tumor therapy, death, withdrawal of Informed Consent Form by subject, early study termination of subject determined by the investigator, or loss to follow-up of subject.

Pharmacokinetics (PK) analysis: For all subjects in each dose group of different cohorts, the drug concentration data at each time point are analyzed separately, and the individual serum concentration-time curves (C-T curves) and mean concentration-time curves (C-T curves) of subjects are provided. The WinNonlin software and non-compartmental model are used to estimate and analyze the PK parameters and calculate the main PK parameters.

Immunogenicity evaluation: Descriptive statistical analysis will be performed for changes in anti-drug antibodies produced of each subjects at each time point after starting treatment.

According to the data as collected by now, IMM01 does not cause obvious hemagglutination, and thus IMM01 administration requires no priming dose or dose ramp-up administrations, making the combination therapy of the disclosure more safe and more convenient. Further, IMM01 has a high binding affinity to the CD47-expressing tumor cells, no more than 2.0 mg/kg of IMM01 can produce single agent in vivo efficacy.

Most importantly, the IMM01+azacitidine combination were well tolerated by CMML and MDS patients with minimal grade 3 and 4 treatment-related hemolysis or minimal treatment discontinuation due to treatment-related adverse reactions, and produced higher CR rate and ORR than the azacitidine monotherapy, suggesting the addition of IMM01 to azacitidine significantly improved the efficacy in CMML and MDS treatment.

The present disclosure is now further described with the non-limiting examples below.

### EXAMPLES

### Example 1. IMM01 (Timdarpacept) Does not Bind to Human red Blood Cells

To test the binding of IMM01, the bifunctional protein consisting of SEQ ID NO: 1, to red blood cells from people of different genders, ages and blood types, 100healthy donors' blood samples were collected. In specific, the donors included 62males and 38 females, with 29 with type A blood, 31 with type B blood, 10 with type AB blood and 30 with type O blood.

The red blood cells were isolated from the blood samples. Red blood cells were diluted with PBS, to a density of about 3×10⁶ cells/mL. The TTI-621 (a SIRPα-IgG1 Fc fusion protein, prepared in house with the sequence from PCT/CA2013/001046), IMM01, hB6H12 (anti-CD47 mAb Fab, variable region sequences from the NCBI database PDB) and hIgG1-Fc (as a negative control) proteins were respectively diluted to 10000 nM in 1% BSA-PBS. Then, 100 μL of the diluted red blood cells were added into 96-well plates, well mixed with 100 μL of TTI-621, IMM01, hB6H12 or hIgG1-Fc, and incubated at 4° C. for 45 min.After incubation, the cells were centrifuged at 260 g for 3 min, the supernatant in each well was discarded, and 200 μL 1% BSA-PBS was added for washing the cells twice with centrifugation at 260 g for 3 min. The secondary antibody, i.e., Anti-Human IgG (Fc)-FITC, was 1:250 diluted, and 100 μL of the resultant secondary antibody solution was added to each well. The 96-well plates were incubated at 4° C. for 45 min, and washed with 100 μL 1% BSA-PBS with centrifugation at 260 g for 3 min. The supernatant from each well was discarded, and the cells were washed with 200 μL of 1% BSA-PBS once with centrifugation at 260 g for 3 min. The cells were re-suspended in 200 μL of 1% BSA-PBS, and subjected to fluorescence signal detection by flow cytometry. The data were analyzed with GraphPad Prism.

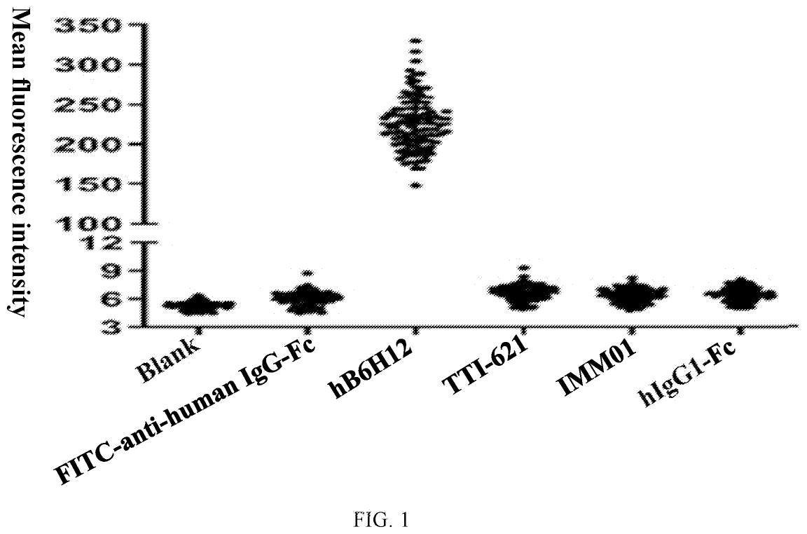

The results were shown in FIG. 1. It can be seen that the conventional anti-CD47 antibody hB6H12 strongly bound to red blood cells, while IMM01 and TTI-621, both of which adopted SIRPα-Fc ligand trap design, showed very weak binding to red blood cells.

### Example 2. IMM01 Does not Induce Hemaglutination ex Vivo

To test whether or not the IMM01 protein agglutinates human red blood cells, blood samples were collected from 8 adults of 18-35 years covering both genders and all four blood types and used in testing.

Briefly, 70 μL human whole blood was added with 200 μL OBS solution, centrifuged at 12000 rpm at room temperature for 3 min, added with 200 μL PBS, centrifuged at 12000 rpm at room temperature for 3 min, and washed twice. Approximately 60 μL of the bottommost red blood cell was collected, added with 6 mL PBS, and turned upside down, to obtain approximately 1% (v/v) human red blood cell suspension.

The TTI-621, IMM01, hB6H12 and hIgG1-Fc proteins were respectively diluted to 8000 nM, and then serially diluted by 3 folds with PBS. The PBS buffer was used as the blank control.

To a coagulation plate, were added 1% (v/v) human red blood cell suspension at 50 μL/well, and the serially diluted proteins at 50 μL/well.

The resultant mixtures were kept still at room temperature for 2 h, to observe the hemagglutination. Photographs were taken at the end of the incubation for recording. The extent of hemagglutination was scored on a scale of 1 to 6, with 1 representing the absence of hemagglutination and 6 representing complete hemagglutination.

According to the results, no hemagglutination occurred when the red blood cells were treated with IMM01 in the concentration range of 0.02 nM-4000 nM, regardless of the gender, age and blood type of the donor from which the blood samples were collected.

In contrast, the conventional anti-CD47 antibody hB6H12, at the concentration of about 5-150 nM, caused obvious hemagglutination, regardless of the gender, age and blood type of the donors from which the blood samples were collected.

### Example 3. Phase I Clinical Trial of IMM01+Azacitidine Combination for Previously Untreated CMML and MDS Patients

The traditional 3+3 design was used for dose escalation and determination of maximum tolerated dose (MTD) and recommended phase 2 dose (RP2D) of the IMM01+azacutudube (AZA) combination.

Briefly, according to the phase I clinical trial design, the IMM01 protein (timdarpacept) in 0.9% physiological saline solution was administrated via intravenous infusion at the dose of 1.0 mg/kg, 1.5 mg/kg or 2.0 mg/kg, once a week, 28 days as a treatment cycle, up to 12 treatment cycles, and azacitidine (VIDAZA®, Bristol myers squibb) was administered subcutaneously at a fixed dose of 75 mg/m²/day for 7 consecutive days in a 28-day cycle, up to 12 treatment cycles. The administration was stopped when a patient experienced intolerable toxicity, disease progression, loss to follow-up, death, withdrawal of consent, discontinuation of treatment in the best interest of the patient in the opinion of the investigator, receipt of other anti-tumor therapy, 48 weeks of treatment, or early termination of the study, whichever occurred first.

Bone marrow cytology was performed before Day 1 of Cycle 2 (time window of—7 days), and efficacy assessment during the treatment period did not affect the subjects' continuation of treatment. Patients with MDS or CMML were examined and assessed every treatment cycle if they did not achieve complete response (CR), or marrow complete response (mCR) with hematological improvement (HI); patients were double checked before the next treatment cycle and assessed every 2 treatment cycles (8 weeks [window: −7 days]) thereafter, if CR or mCR+HI was achieved; and patients were examined and assessed every treatment cycle or at the investigator's discretion if mCR was achieved. Patients were closely observed for adverse events from the start of the first dose until 28 days after the last dose (window: ±7 days). Care was taken to avoid the use of granulocyte colony-stimulating factor, blood transfusion or other behaviors that may affect the results during efficacy evaluation.

The phase I trial was ended upon occurrence of the last subject withdrawing consent, terminating treatment or withdrawing from the trial, lost to follow-up or death, being treated for 48 weeks or ending the study prematurely, whichever occurred first.

MTD is defined as the maximum dose at which the incidence of dose-limiting toxicity (DLT) is <⅓. For the dose level to be determined as MTD, there must be at least 6 subjects with evaluable DLT data. Once MTD is determined, MTD is usually used as RP2D, or the dose level below MTD can be selected as RP2D.

RP2D was initially determined based on the safety, PK, efficacy, and other data of dose escalation in the combination therapy, in combination with the safety, tolerability and PK/PD data of the dose escalation and dose expansion in IMM01 monotherapy, and finally decided by the scientist review committee (SRC).

Through careful dose escalation, balancing efficacy with adverse events, pharmacokinetics and pharmacodynamics, the dosing regimen of IMM01 at 2.0 mg/kg was chosen as the recommended phase 2 dose (RP2D) for previously untreated CMML and MDS patients.

### Example 4. Phase II Clinical Trial of IMM01+Azacitidine Combination for Previously Untreated CMML and MDS Patients

In the phase II clinical trial, patients were and are being enrolled according to the inclusion and exclusion criteria described in the DETAILED DESCRIPTION OF THE INVENTION part above.

The patients, once enrolled, were administered with IMM01 (timdarpacept) intravenously at 2.0 mg/kg once a week, with every 28 days as a treatment cycle, and with azacitidine at 75 mg/m²/day once daily subcutaneously, with each 28-day treatment as one treatment cycle. On days when both IMM01 and azacitidine were administered, the azacitidine administration was about 65 minutes to about 75 minutes after the IMM01 injection.

### Example 5. Safety and Efficacy of IMM01 (Timdarpacept) Together With Azacitidine in Previously Untreated CMML Patients

The baseline characteristics of the CMML patients enrolled in phase IIa trial were summarized in Table 5. Notably, 60.9% (14 out 23) of the patients had high risk based on CPSS-mol classification (Elena C, et al., (2016) Blood. 128(10):1408-17). A substantial proportion of patients had significant aberrant baseline hematologic conditions.

TABLE 5
Baseline characteristics of the previously untreated
CMML patients in phase IIa

| | Previously untreated |
|---|---|
| Characteristics | CMML (N = 23) |
| Median age, yrs (range) | 62 (38-75) |
| Gender | |
| Male | 15 (65.2%) |
| Female | 8 (34.8%) |
| ECOG Performance Status, n (%) | |
| 0 | 6 (26.1%) |
| 1 | 15 (65.2%) |
| 2 | 2 (8.7%) |
| CPSS-mol classification | |
| Intermediate risk-1 (1) | 0 |
| Intermediate risk-2 (2-3) | 6 (26.1%) |
| High risk (≥4.0) | 14 (60.9%) |
| unknow | 3 (13.0%) |
| Subtype (2016 WHO) | |
| CMML-1 | 12 (52.2%) |
| CMML-2 | 11 (47.8%) |
| Subtype (FAB) | |
| Myelodysplastic-CMML | 7 (30.4%) |
| (MD-CMML) | |
| Myeloproliferative-CMML | 14 (60.9%) |
| (MP-CMML) | |
| unknow | 2 (8.7%) |
| Baseline Hematology, median (range) | |
| White blood cells (10⁹/L) | 8.4 (2.9, 17.2) |
| Neutrophil (10⁹/L) | 4.7 (0.9, 11.9) |
| Platelets (10⁹/L) | 66 (5, 667) |
| Hemoglobin (g/L) | 69 (32, 132) |

Table 6 summarized the efficacy data in phase IIa as recorded as of May 19, 2023. It can be seen that the complete response rate (CR) from the CMML patients in the trial increased over time and reached 50% (3 out of 6) after 4 months of treatment. Likewise, objective response rate (ORR), defined as the sum of complete response (CR) rate, marrow complete response (mCR) rate, marrow complete response with hematological improvement (mCR+HI) rate and hematological improvement (HI) rate, also increased over time and reached 100% (6 out of 6) after 4 months of treatment.

TABLE 6
Efficacy of IMM01 + AZA combination in previously untreated CMML
patients in phase IIa
Best Overall Response in Previously untreated CMML (n, %)

| | Evaluable | | | |
|---|---|---|---|---|
| | analysis set | ≥2 months | ≥3 months | ≥4 months |
| | (N = 20) | (N = 14) | (N = 12) | (N = 6) |
| ORR | 11 (55%) | 11 (78.6%) | 10 (83.3%) | 6 (100%) |
| CR | 4 (20%) | 4 (28.6%) | 4 (33.3%) | 3 (50.0%) |
| mCR + HI | 2 (10%) | 2 (14.3%) | 2 (16.7%) | 1 (16.7%) |
| mCR alone | 5 (25%) | 5 (35.7%) | 4 (33.3%) | 2 (33.3%) |
| HI alone | 0 | 0 | 0 | 0 |
| SD | 2 (10%) | 2 (14.3%) | 2 (16.7%) | 0 |
| SD*(NE) | 4 (20%) | 1 (7.1%) | 0 | 0 |
| PD | 3 (15%) | 0 | 0 | 0 |

SD: Stable Disease;
PD: Progressive Disease;
NE: Not Evaluable.

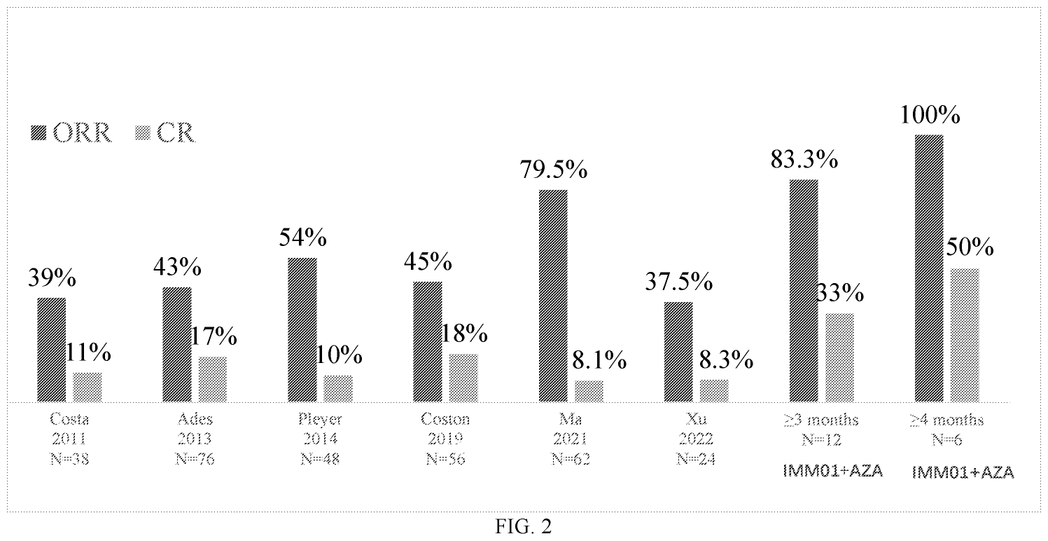

In comparison, as shown in FIG. 2, CMML CR rate and ORR in multiple retrospective clinical studies, various CMML trials and two Chinese real world studies of hypomethylating agents (including 5-azacitidine) did not reach 20% and 80%, respectively (Costa et al., (2011) Cancer. 117(12):2690-6; Ades et al., (2013) Leuk Res. 37(6):609-13; Pleyer et al. (2014) Leuk Res. 38(4):475-83; Coston et al., (2019) Am J Hematol. 94(7):767-779; Ma et al., (2021) Cancer Med. 10(5):1715-1725; Xu et al., (2022) Invest New Drugs. 40(5):1117-1124).

Therefore, the addition of IMM01 (timdarpacept) to azacitidine significantly improved the efficacy in CMML treatment.

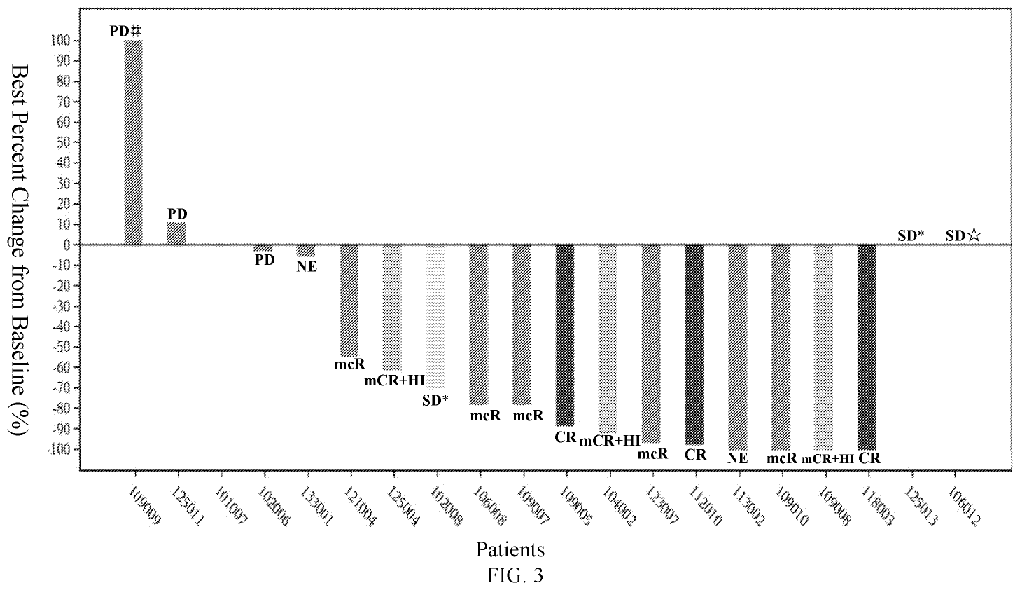

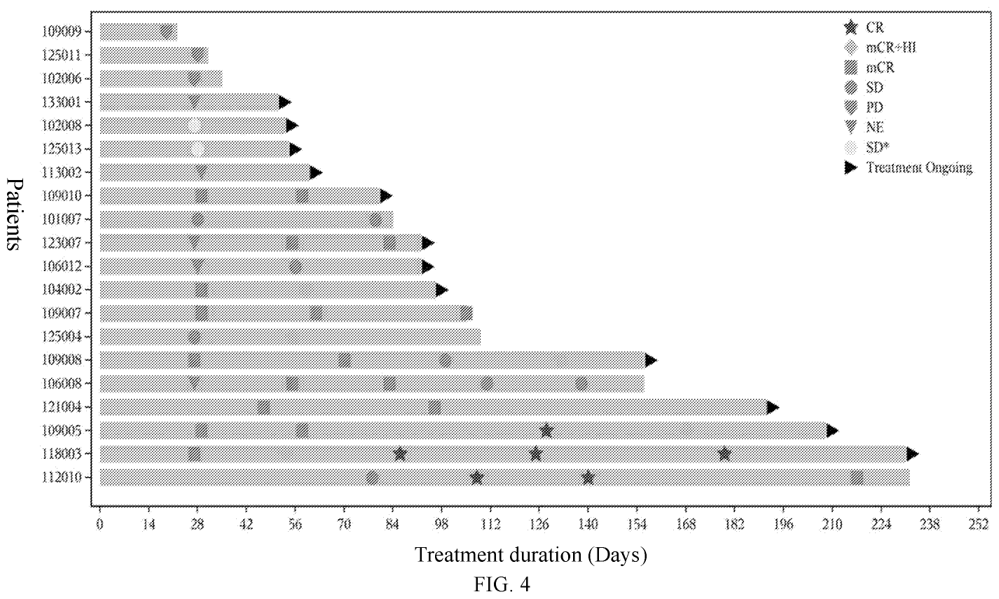

The CMML patients' individual best percentage change in bone marrow blast from baseline and duration of response were shown in FIG. 3 and FIG. 4, respectively.

Treatment related adverse events (TRAEs) of the combination of IMM01(timdarpacept) and azacitidine for CMML were summarized in Table 7. Overall, the combination was well tolerated in previously untreated CMML patients.

TABLE 7
Treatment related adverse events in IMM01 + AZA combination
therapy in previously untreated CMML patients in phase IIa

| | Previously untreated CMML | |
|---|---|---|
| | (N = 10) n, (%) | |
| TRAE (≥20%) | All Grades | G3-4 |
| ≥1 TRAEs | 10 ( 100) | 8 (80.0) |
| Hematological | | |
| Leukopenia | 8 (80.0) | 6 (60.0) |
| Lymphopenia | 7 (70.0) | 6 (60.0) |
| Neutropenia | 7 (70.0) | 5 (50.0) |
| Thrombocytopenia | 5 (50.0) | 5 (50.0) |
| Anemia | 4 (40.0) | 1 (10.0) |
| leukocytosis | 2 (20.0) | 0 |
| Elevated Neutrophil | 2 (20.0) | 0 |
| Count | | |
| Other | | |
| Infusion related | 5 (50.0) | 0 |
| reaction | | |
| Constipation | 3 (30.0) | 0 |
| Nausea | 3 (30.0) | 0 |
| Fatigue | 2 (20.0) | 1 (10.0) |
| Hyperbilirubinemia | 2 (20.0) | 1 (10.0) |
| Pyrexia | 2 (20.0) | 0 |
| Vomiting | 2 (20.0) | 0 |
| Appetite loss | 2 (20.0) | 0 |
| Hypoalbuminaemia | 2 (20.0) | 0 |
| Hyponatraemia | 2 (20.0) | 0 |
| C-reactive protein | 2 (20.0) | 0 |
| elevated | | |
| Procalcitonin elevated | 2 (20.0) | 0 |
| Pleural effusion | 2 (20.0) | 0 |
| Infection | 2 (20.0) | 0 |

Most commonly reported TRAE above 20% within all grades and in grades above 3 were hematological events. As of Feb. 10^{th} 2023, no hemolytic anemia or treatment-related grade 3 and 4 hemolysis was observed. Only one patient (10%) discontinued treatment because of TRAEs. The most common Grade 3 or 4 TRAEs occurred in ≥20% were also frequently reported in MDS patients treated with azacitidine monotherapy in China (Xin Du et al., (2017) Asia Pac J Clin Oncol. 14(3):270-278).

### Example 6. Safety and Efficacy of IMM01 (Timdarpacept) Together With Azacitidine in Previously Untreated MDS Patients

TABLE 8
Baseline characteristics of previously untreated MDS
patients in phase IIa

| Characteristics | IMM01 + AZA (N = 51) |
|---|---|
| Median age, yrs (range) | 64 (32-83) |
| Gender | |
| Male | 37 (72.5%) |
| Female | 14 (27.5%) |
| ECOG Performance Status, n (%) | |
| 0 | 2 (3.9%) |
| 1 | 41 (80.4%) |
| 2 | 8 (15.7%) |
| IPSS-R classification | |
| Intermediate risk (>3.0-≤4.5) | 12 (23.5%) |
| High risk (>4.5-≤6.0) | 25 (49.0%) |
| Very High risk (>6.0) | 13 (25.5%) |
| Unknown | 1 (2.0%) |
| 2016 WHO of MDS (Subtype) | |
| MDS with single lineage dysplasia (MDS-SLD) | 0 |
| MDS with multilineage dysplasia (MDS-MLD) | 5 (9.8%) |
| MDS with ring sideroblasts (MDS-RS) | 1 (2.0%) |
| MDS with excess blasts-1 (MDS-EB-1) | 20 (39.2%) |
| MDS with excess blasts-1 (MDS-EB-2) | 17 (33.3%) |
| MDS, unclassifiable (MDS-U) | 2 (3.9%) |
| Unknown | 6 (11.8%) |
| Baseline Hematology, median (range) | |
| White blood cells (10⁹/L) | 2.1 (0.6, 12.5) |
| Neutrophil (10⁹/L) | 0.7 (0.1, 8.6) |
| Platelets (10⁹/L) | 36 (2, 409) |
| Hemoglobin (g/L) | 69 (35, 95) |

Baseline characteristics of the MDS patients as enrolled were summarized in Table 8. Of note, 49.0% (25 out 51) of the MDS patients had high risk and 25.5% (13 out of 51) of the patients had very high risk based on IPSS-R classification (Greenberg PL, et al., (2012) Blood. 120(12):2454-65). A substantial proportion of patients had significant aberrant baseline hematologic conditions.

Table 9 summarized the efficacy data in phase IIa as recorded as of May 19, 2023. As can be seen, CR from the MDS patients in the trial increased over time and reached 28.6% (6 out of 21) after treatment of 4 months or more and 35.3% (6 out of 17) after treatment of 6 months or more. Similarly, ORR, defined as the sum of CR rate, marrow complete response (mCR) rate, marrow complete response with hematological improvement (mCR+HI) rate and hematological improvement (HI) rate, also increased over time and reached 81.0% (17 out of 21) after treatment of 4 months or more and 88.2% (15 out of 17) after treatment of 6 months or more.

TABLE 9
Efficacy of IMM01 + AZA combination in previously
untreated DMS patients in phase IIa

| | Time Since First Dose (ES N = 40) | | | |
|---|---|---|---|---|
| Best Overall | ≥1 month | ≥3 months | ≥4 months | ≥6 months |
| Response | (N = 40) | (N = 30) | (N = 21) | (N = 17) |
| ORR | 23 (57.5%) | 21 (70.0%) | 17 (81.0%) | 15 (88.2%) |
| CR | 6 (15.0%) | 6 (20.0%) | 6 (28.6%) | 6 (35.3%) |
| mCR + HI | 8 (20.0%) | 8 (26.7%) | 6 (28.6%) | 6 (35.3%) |
| mCR alone | 7 (17.5%) | 5 (16.7%) | 3 (14.3%) | 2 (11.8%) |
| HI | 2 (5.0%) | 2 (6.7%) | 2 (9.5%) | 1 (5.9%) |
| SD | 11 (27.5%) | 8 (26.7%) | 4 (19.0%) | 2 (11.8%) |
| NE (SD*) | 4 (10.0%) | 1 (3.3%) | 0 | 0 |
| PD | 2 (5.0%) | 0 | 0 | 0 |

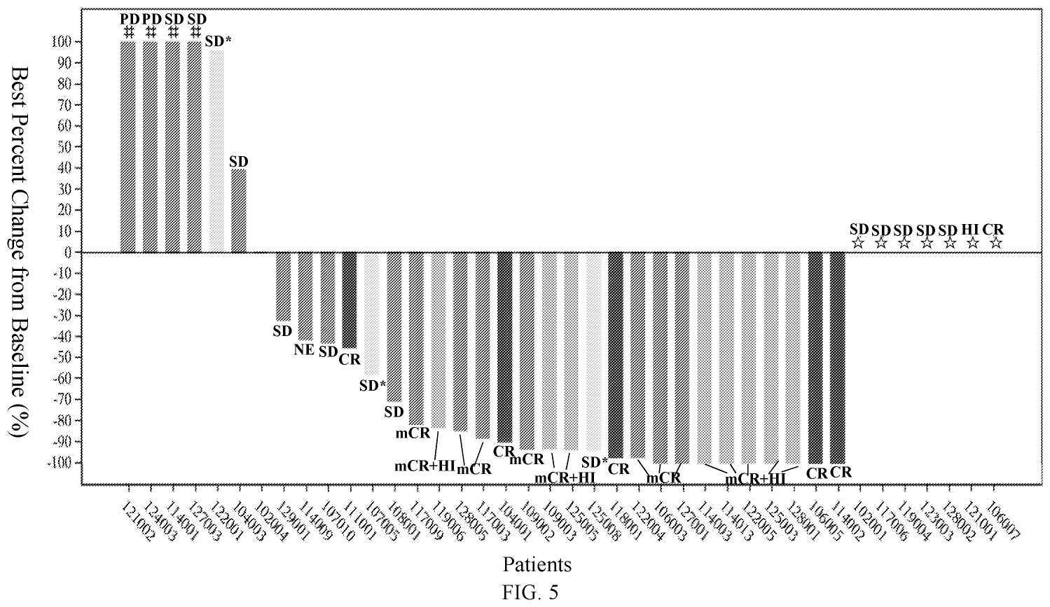

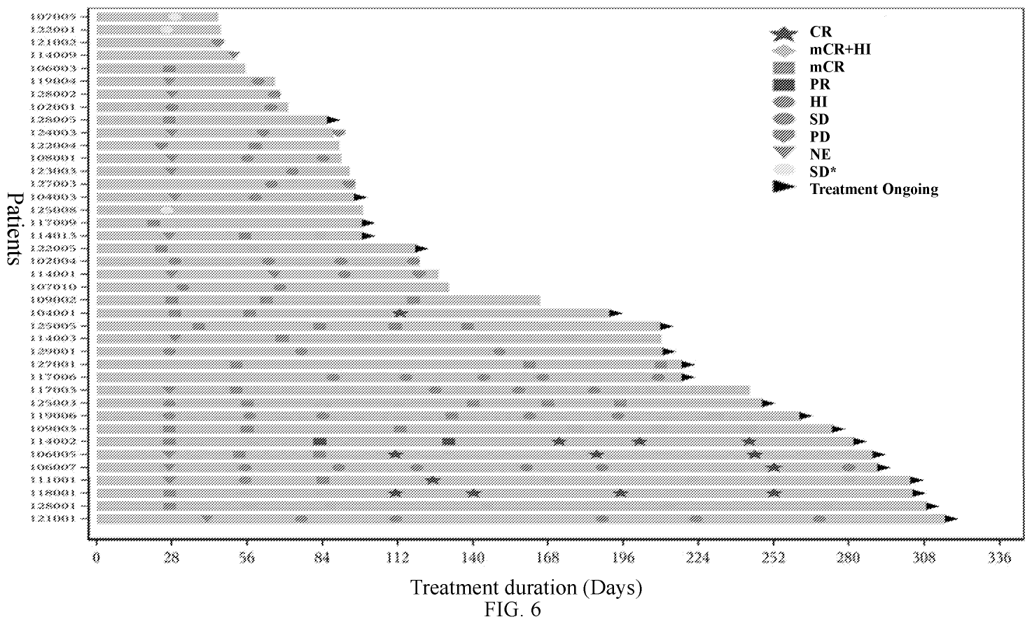

The MDS patients' individual best percentage change in bone marrow blast from baseline and duration of response were shown in FIG. 5 and FIG. 6, respectively.

Table 10 summarized the TRAEs occurred in the combination of IMM01(timdarpacept) and azacitidine in MDS patients. Overall, the combination was well tolerated in previously untreated MDS patients. Most commonly reported TRAE above 20% within all grades and in grades above 3 were hematological events. As of Feb. 10^{th} 2023, no hemolytic anemia, treatment-related grade 3 and 4 hemolysis or treatment discontinuation due to TRAEs was observed. The most common Grade 3 or 4 TRAEs occurred in ≥20% were also frequently reported in MDS patients treated with azacitidine monotherapy in China (Xin Du et al., (2017) supra).

TABLE 10
Treatment related adverse events in IMM01 + AZA combination therapy in
previously untreated MDS patients in phase IIa

| | Previously untreated MDS (N = 44) | |
|---|---|---|
| | n, (%) | |
| TRAE (≥20%) | All Grades | G3-4 |
| ≥ITRAEs | 42 (95.5) | 38 (86.4) |
| Hematological | | |
| Leukopenia | 31 (70.5) | 29 (65.9) |
| Thrombocytopenia | 29 (65.9) | 25 (56.8) |
| Neutropenia | 26 (59.1) | 26 (59.1) |
| Lymphopenia | 20 (45.5) | 19 (43.2) |
| Anemia | 21 (47.7) | 21 (47.7) |
| Other | | |
| Vomiting | 19 (43.2) | 0 |
| Pyrexia | 15 (34.1) | 1 (2.3) |
| Infusion related | 14 (31.8) | 2 (4.5) |
| reaction | | |
| Nausea | 13 (29.5) | 0 |
| Constipation | 11 (25.0) | 1 (2.3) |
| Infection | 10 (22.7) | 8 (18.2) |
| Hypoalbuminaemia | 9 (20.5) | 0 |

SEQ ID NO: 1
EEELQVIQPDKSVSVAAGESAILHCTVTSLIPVGPIQWFRGAGPARELIY
NQKEGHFPRVTTVSESTKRENMDFSISISAITPADAGTYYCVKFRKGSPD
TEFKSGAGTELSVRAKPSAPVVSGPAARATPQHEPKSCDKTHTCPPCPAP
ELLGGPSVFLFPPKPKDTLMISRTPEVTCVVVDVSHEDPEVKFNWYVDGV
EVHNAKTKPREEQYNSTYRVVSVLTVLHQDWLNGKEYKCKVSNKALPAPI
EKTISKAKGQPREPQVYTLPPSRDELTKNQVSLTCLVKGFYPSDIAVEWE
SNGQPENNYKTTPPVLDSDGSFFLYSKLTVDKSRWQQGNVFSCSVMHEAL
HNHYTQKSLSLSPGK
SEQ ID NO: 2
EEELQVIQPDKSVSVAAGESAILHCTVTSLIPVGPIQWFRGAGPARELIY
NQKEGHFPRVTTVSESTKRENMDFSISISAITPADAGTYYCVKFRKGSPD
TEFKSGAGTELSVRAKPSAPVVSGP
SEQ ID NO: 3
AARATPQHEPKSCDKTHTCPPCPAPELLGGPSVFLFPPKPKDTLMISRTP
EVTCVVVDVSHEDPEVKFNWYVDGVEVHNAKTKPREEQYNSTYRVVSVLT
VLHQDWLNGKEYKCKVSNKALPAPIEKTISKAKGQPREPQVYTLPPSRDE
LTKNQVSLTCLVKGFYPSDIAVEWESNGQPENNYKTTPPVLDSDGSFFLY
SKLTVDKSRWQQGNVFSCSVMHEALHNHYTQKSLSLSPGK

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

We claim:

1. A method of treating chronic myelomonocytic leukemia in a subject in need thereof, comprising the steps of:
(a) administering intravenously to the subject for the chronic myelomonocytic leukemia about 2.0 mg/kg body weight per day of a recombinant fusion protein in the form of a composition comprising a pharmaceutically acceptable excipient and the recombinant fusion protein,
(b) about 65 minutes to about 75 minutes after completing the administering of step (a), administering subcutaneously to the subject about 75 mg/m² of azacitidine, and
(c) after steps (a) and (b), repeating step (a) once weekly, and administering subcutaneously to the subject about 75 mg/m² of azacitidine once daily, wherein on the days that the subject is also having step (a) repeated, the azacitidine is administered about 65 minutes to about 75 minutes after completing the repeated administering of step (a),
wherein the recombinant fusion protein consists of SEQ ID NO: 1 that includes a mutated SIRP-alpha D1 domain and a functional IgG1 heavy chain constant region, and wherein the administering does not include administering a priming dose of the recombinant fusion protein or dose ramp-up administrations of the recombinant fusion protein to mitigate on-target anemia.

2. The method of treating chronic myelomonocytic leukemia of claim 1, wherein after 16 weeks or longer of the treating, the treating produces more than 85%, 90% or 95% of objective response rate, wherein the objective response rate is defined as the sum of complete response rate, marrow complete response rate, marrow complete response with hematological improvement rate and hematological improvement rate.

3. The method of treating chronic myelomonocytic leukemia of claim 1, wherein after 16 weeks or longer of the treating, the treating produces more than 30%, 35%, 40%, or 45% of complete response rate.

4. The method of treating chronic myelomonocytic leukemia of claim 1, wherein no more than 5% or 10% of subjects receiving the administering of step (a) and the repeated administering of step (c) have chance of treatment-related hemolytic anemia.

5. The method of treating chronic myelomonocytic leukemia of claim 1, wherein not more than 5% or 10% of subjects receiving the of step (a) and the repeated administering of step (c) have chance of treatment-related grade 3 and 4 hemolysis.

6. The method of treating chronic myelomonocytic leukemia of claim 1, wherein not more than 15% or 20% of subjects receiving the of step (a) and the repeated administering of step (c) have chance of treatment discontinuation due to treatment-related adverse effects.

7. A method of treating myelodysplastic syndrome in a subject in need thereof, comprising the steps of:
(a) administering intravenously to the subject for the myelodysplastic syndrome about 2.0 mg/kg body weight per day of a recombinant fusion protein in the form of a composition comprising a pharmaceutically acceptable excipient and the recombinant fusion protein,
(b) about 65 minutes to about 75 minutes after completing the administering of step (a), administering subcutaneously to the subject about 75 mg/m² of azacitidine, and
(c) after steps (a) and (b), repeating step (a) once weekly, and administering subcutaneously to the subject about 75 mg/m² of azacitidine once daily, wherein on the days that the subject is also having step (a) repeated, the azacitidine is administered about 65 minutes to about 75 minutes after completing the repeated administering of step (a),
wherein the recombinant fusion protein consists of SEQ ID NO: 1 that includes a mutated SIRP-alpha D1 domain and a functional IgG1, and
wherein the administering of step (a) and the repeated administering of step (a) do not include administering a priming dose of the recombinant fusion protein or dose ramp-up administrations of the recombinant fusion protein to mitigate on-target anemia.

8. The method of claim 7, wherein after 16 weeks or longer of the treating, the treating produces more than 70%, 75% or 80% of objective response rate, wherein the objective response rate is defined as the sum of complete response rate, marrow complete response rate, marrow complete response with hematological improvement rate and hematological improvement rate.

9. The method of claim 7, wherein after 16 weeks or longer of the treating, the treating produces more than 20% or 25% of complete response rate.

10. The method of claim 7, wherein no more than 5% or 10% of subjects receiving the administering of step (a) and the repeated administering of step (c) have chance of treatment-related hemolytic anemia.

11. The method of treating myelodysplastic syndrome of claim 7, wherein not more than 5% or 10% of subjects receiving the of step (a) and the repeated administering of step (c) have chance of treatment-related grade 3 and 4 hemolysis.

12. The method of treating myelodysplastic syndrome of claim 7, wherein not more than 5%, 10%, 15% or 20% of subjects receiving the of step (a) and the repeated administering of step (c) have chance of treatment discontinuation due to treatment-related adverse effects.


SEQUENCE LISTING (3 sequences)

SEQ ID NO: 1 - protein, 365 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGPAARATPQHEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 125 aa, synthetic construct
EEELQVIQPDKSVSVAAGESAILHCTVTSLIPVGPIQWFRGAGPARELIYNQKEGHFPRVTTVSESTKRENMDFSISISAITPADAGTYYCVKFRKGSPDTEFKSGAGTELSVRAKPSAPVVSGP

SEQ ID NO: 3 - protein, 240 aa, synthetic construct
AARATPQHEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK